(19) United States
(12) Patent Application Publication
(10) Pub. No.: US 2008/0183107 A1
(43) Pub. Date: Jul. 31, 2008

(54) Apparatus and Method for Measurement of Force Exerted by the Tongue and the Lips and Their Applications in Tests and Therapies

(76) Inventors: Bruce D. Miller, North Reading, MA (US); Diana Pehlman-Wilkin, Plano, TX (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/668,391

(22) Filed: Jan. 29, 2007

(51) Int. Cl. A61B 5/103

(52) U.S. Cl. 600/590; 128/897

(57) ABSTRACT

An apparatus for measurement of force exerted by the tongue or the lips. The apparatus comprises a sensor assembly having a force-receiving element, a force transducer providing a sensor signal, and a shaft mechanically coupling the force applied to the force-receiving element to the force transducer. An apparatus for providing isometric or resistive treatment to increase the strength and motility of the tongue or the lips. The apparatus comprises an isometric assembly or a resistive assembly having a force-receiving element providing an isometric barrier or a resistive force, respectively, a support structure, and a shaft connecting the force-receiving element to the support structure.

### BACKGROUND

Neurological changes to the cranial nerves innervating the muscles of the oral cavity can result in decreased muscle tone, strength, and coordination of movement. This can cause medical conditions known as Dysarthria, oral Dysphagia, and oral Apraxia. These conditions affect the motor and sensory systems of the oral mechanisms (lips, tongue, and palate). Causes can be degenerative or non-degenerative. Non-degenerative lesions or damage can include Cerebrovascular Accident (ischemic or hemorrhagic), Traumatic Brain injury, and Brain Tumor. Examples of degenerative conditions include Parkinson's Disease, Amyotrophic Lateral Sclerosis, Multiple Sclerosis, and Myasthenia Gravis.

Dysarthria involves impairment of muscle control affecting the speech systems of respiration, phonation, resonance, articulation, and prosody. This results in decreased precision, intelligibility and naturalness of speech output. Oral Dysphagia is an impairment of the oral musculature and oral stage of swallowing. Deficits can include restricted mastication, reduced oral containment of food and liquid, poor lingual bolus formation/propulsion, and delayed initiation of the swallow reflex. Medical complications can include insufficient nourishment and hydration. Oral Apraxia is the impairment of volitional motor movements of the oral mechanisms (lips, tongue, and palate). This can result in restricted speech output and impaired oral phase of swallowing.

These medical conditions are typically treated by a Speech-Language Pathologist. Treatment modalities may include range of motion and isometric exercises of the oral mechanisms. These techniques facilitate coordination and strength of the speech and swallowing muscles. Labial and lingual isometric exercises are completed with use of a tongue depressor. Strengthening is accomplished by repetitive isometric force against the tongue depressor. This is completed in the lateral directions (side to side), protrusion/extension (out), upward and downward directions. Labial isometric exercises can also include completion of a strong labial seal on the tongue depressor. Diagnostic baseline data and therapeutic progress is measured by a subjective and qualitative assessment of force against the tongue depressor by the Speech Pathologist. This measurement technique is very inaccurate as the perception of the exerted force is inexact and varies from session to session. In addition, patients may be treated by different Speech Pathologists from session to session with overall progress subjectively assessed by several clinicians. Given variance in perception of force from each individual Speech Pathologist the measurement is inexact.

FIG. 1 shows a pictorial view of the current therapeutic technique for improving the strength and motility of the tongue. A tongue depressor is used to provide an isometric boundary and the therapist will make a subjective determination of force based upon the amount of force required by them to keep the tongue depressor static. The patient 2 presses with his/her tongue 3 against a tongue depressor 4 while the Speech-Language Pathologist or other care giver 5 creates enough opposing force to create positional equilibrium. The perceived amount of force will be recorded and used as a metric of strength and rehabilitative progress. In FIG. 1, the measurement and therapy is being conducted in the direction of extension (outward). However, the same technique is used for lateral (side to side) and vertical (up and down) directions. There are similar mechanisms for measuring labial strength. The tongue depressor can be placed right in front of one or both lips while the patient presses with his/her upper, lower, or both lips against it, exerting a forward force such as in forming a long “u” sound. In addition, labial strength can be measured by having the patient create a seal against the tongue depressor with his/her lips while it is inserted into the mouth. The therapist will try to extract the tongue depressor and measure the amount of force required to break the labial seal with the tongue depressor.

### SUMMARY

The present invention provides apparatus and method for measurement of force exerted by the tongue and the lips and their applications in tests and therapies. The apparatus is economical to manufacture, the method is easy and efficient to operate, allowing repeatable measurement of the tongue or the lips in multiple directions and providing more objective measurement under conditions of clinical use. In addition to performing measurement in tests, the apparatus and method can be applied to rehabilitative medicine for use with patients requiring isometric or resistive therapy to improve the strength and motility of the tongue or the lips.

Briefly, apparatus for measuring the strength or motility of the tongue or the lips comprises a sensor assembly comprising a force-receiving element, which can be a plate, a force transducer providing a sensor signal, and a shaft with either fixed or adjustable length mechanically coupling the force applied to the force-receiving element to the force transducer. A disposable element can be either integral to, or attachable to the force-receiving element. The sensor assembly can be mounted to a disk so as to allow measurement in multiple directions. Sensor signals generated by the force transducer can be processed by electronics to produce an output signal, which can comprise a profile of the amplitude of the force exerted by the tongue or the lips over time.

Apparatus for providing therapy to increase the strength and motility of the tongue or the lips comprises an assembly comprising a force-receiving element, which can be a plate, a support structure, and a shaft with fixed or adjustable length connecting the force-receiving element to the support structure. A disposable element can be either integral to, or attachable to the force-receiving element. The assembly can be mounted to a disk so as to allow treatment in multiple directions. It can include a spring surrounding a guiding element attachable to the force-receiving element and captured in a tube, wherein the tube is connected to the shaft. It can also include a force transducer providing a signal, which can be processed by electronics to produce an output signal, which can comprise a profile of the amplitude of the force exerted by the tongue or the lips over time.

Method of measuring the strength or motility of the tongue or the lips comprises contracting the muscles of the tongue or the lips to exert force on a force-receiving element disposed in front of the patient by either pressing against the force-receiving element with the patient's tongue or lips, or by creating a labial seal, sensing the force exerted on the force-receiving element by the tongue or the lips to provide an electrical signal representative of the force exerted on the force-receiving element by the tongue or the lips over the duration of a test run, deriving from the electrical signal a profile thereof over the duration of the test run, wherein the profile of the electrical signal is characteristic of the force exerted by the tongue or the lips over the duration of the test run, and displaying and/or recording the profile of force.

Method of providing therapy to increase the strength and motility of a patient's tongue or lips comprises contracting the muscles of the tongue or the lips to exert force on a force-receiving element disposed in front of the patient and connected to a support structure by a shaft. The force-receiving element can either provide an isometric barrier or a resistive force. In the latter situation, the force exerted on the force-receiving element by the tongue or the lips results in displacement of the force-receiving element. Contracting the muscles of the tongue or the lips to exert force on the force-receiving element comprises either pressing against the force-receiving element with the patient's tongue or lips, or creating a labial seal. The force exerted on the force-receiving element can be sensed and transduced into an electrical signal representative of the force exerted on the force-receiving element by the tongue or the lips over the duration of a therapy session. The profile of the electrical signal is characteristic of the force exerted by the tongue or the lips over the duration of the therapy session, and a profile of force corresponding to the characteristic profile of the electrical signal can be displayed and/or recorded over the duration of the therapy session.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a pictorial view of the current therapeutic technique for improving the strength and motility of the tongue, showing a patient using a tongue depressor held by a therapist.

FIG. 2 is an overall pictorial view showing a patient using apparatus according to the invention.

FIG. 3 is a perspective view of an embodiment of a sensor assembly.

FIG. 4 is a perspective view of a spatial array of two sensor assemblies.

FIG. 5A is a middle cross-section of a handheld system comprising a spatial array of two sensor assemblies, one mounted to the center of the front disk and the other mounted to the periphery of the front disk and positioned in the 6:00 o'clock position.

FIG. 5B is a perspective view of a spatial array of two sensor assemblies, one mounted to the center of the front disk and the other mounted to the periphery of the front disk and positioned in the 3:00 o'clock position.

FIG. 6 is a middle cross-section of the handheld system in FIGS. 5A and 5B with a disposable that can create a labial seal, two examples of which are exploded in perspective view.

FIG. 7 is a block diagram of electrical circuitry for operation with apparatus according to the invention, wherein the electrical circuitry is shown connected to a spatial array of two sensor assemblies shown in perspective view.

FIG. 8 is a diagram showing the interface electronics in FIG. 7.

FIG. 9A is a perspective view of an isometric assembly.

FIG. 9B is a perspective view of a resistive assembly.

FIG. 10 are graphs showing the results of three prototypical tests using apparatus according to the invention.

FIG. 11 is a diagram showing a software architecture for an embodiment of apparatus according to the invention.

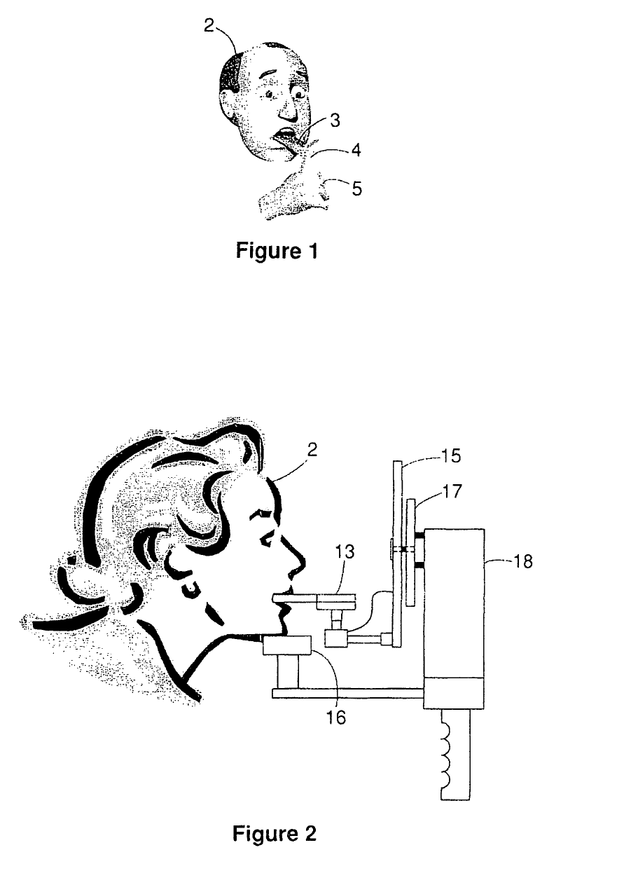

### DETAILED DESCRIPTION

A description of example embodiments of the invention follows.

The present invention is directed to the measurement of lingual strength and motility, labial strength and motility, and provides a mechanism for isometric and resistive strength training. FIG. 2 shows an illustrative embodiment of a measurement apparatus according to the invention. The apparatus includes a sensor assembly 13, which senses the force exerted by the tongue or the lips of the patient 2, whose chin is positioned on a chin support 16. In FIG. 2, the sensor assembly is shown positioned for making a measurement of the patient's tongue muscle strength in a downward direction. The sensor assembly is generally placed in engagement with the muscle to be tested in a manner to be described more fully below. When the patient exerts a force on the sensor assembly, the sensor assembly produces a sensor signal representative of the force of the muscle exerting the force. Sensor assembly 13 is mounted to a disk 15, which is pinned to disk 17 so that disk 15 can freely rotate. Disk 17 is connected to a structural support 18, which can house an electronic system with display, memory and other functions. As illustrated in FIG. 2, the apparatus includes only a single sensor assembly. It may of course also be configured with a plurality of sensor assemblies, which is to be discussed more fully below.

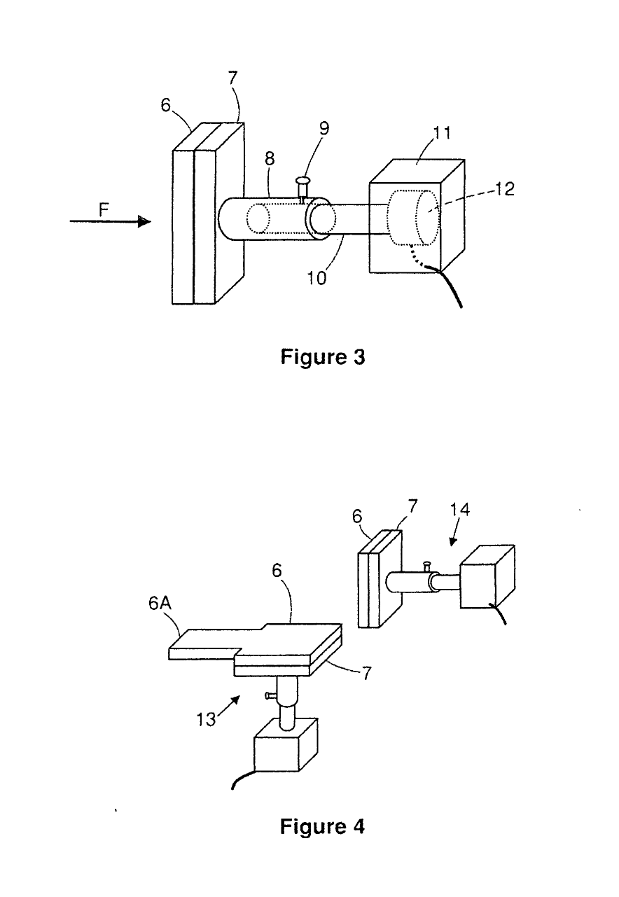

FIG. 3 shows an embodiment of a sensor assembly. In this arrangement, the tongue or the lips presses against a disposable element 6 that is mated with a force-receiving element 7. In this embodiment, the force-receiving element is a metal plate, though other embodiments may use other materials or shapes, or a combination thereof e.g. a plastic cube. The disposable element is typically constructed of plastic and either snaps into the metal plate or can be implemented as a sheath that covers the metal plate 7. The intent of the disposable element is to prevent the carryover of contamination from one patient to another without the need to replace the metal plate 7 or disinfect it between patients. In this embodiment, the disposable element is attachable to the force-receiving element. In other embodiments of the invention, the disposable element can be integral to the force-receiving element. The metal plate assembly can be removable for sterilization on an interval basis. The metal plate 7 is attached to a hollow shaft 8 that mates with shaft 10. Pin 9 is a spring loaded assembly that protrudes through shaft 8 and mates with shaft 10 by inserting into a set of holes (not shown). This mechanism allows the system to have a range of adjustment in order to accommodate different sized patients and measurement of tongue strength at different levels of extension. A spring (not shown) could be used to pretension shaft 8 into shaft 10 and a metal guide (not shown) aid to align the pin 9 with the axis of the holes. Shaft 10 mechanically mates to the force transducer 12 enclosed in housing 11. In this fashion, force applied to the disposable element 6 (indicated by letter “F” in FIG. 3 with an arrow indicating direction of the force) can be mechanically coupled to the force transducer 12.

There are many types of force transducers that have been developed over the years. One of the most commonly used force transducers is known as an “electrical resistive strain gauge”. Conceptually, when a wire is placed into a state of tension within elastic limits, it will increase in length and decrease in diameter. The geometric change modifies the electrical resistance of the wire. In practice, this type of gauge is implemented by creating a metal pattern on a substrate. When a force is applied, the resulting change in resistance can be used to determine the magnitude of that force (provided the gauge has been calibrated to a standard). The techniques used to manufacture this type of transducer are essentially the same as those used in the production of integrated circuits and therefore, this type of transducer can be manufactured at very low cost.

The sensor assembly of FIG. 3 may be arranged in a fashion as shown in FIG. 4. Sensor assembly 14 is intended to measure lingual or labial strength in the direction of extension. Sensor assembly 13 is intended to measure lingual or labial strength in the vertical and lateral directions. Sensor assembly 13 may include a disposable element 6 having an extension portion 6A forward, which can be inserted into the mouth or adjacent to the lips. This allows the patient to press against this element with the side of the tongue or lips without straining for full extension. Extension portion 6A can assume a fixed or adjustable length. Since it is a part of disposable 6, it can also be made in various sizes to accommodate different sized patients.

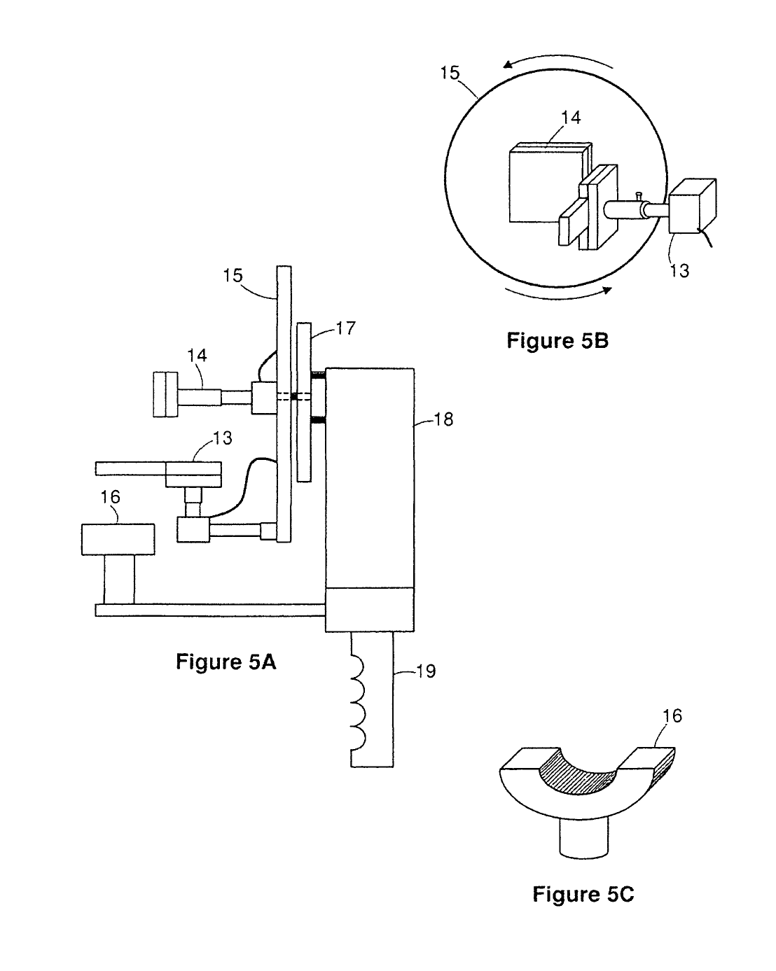

FIGS. 5A and 5B show the system implemented as a handheld device. In this particular embodiment, sensors 13 and 1.4 are mounted to a disk 15. Sensor 14 is arranged in the center of the disk 15 and can be used to measure lingual or labial strength by extension or seal (shown in a subsequent arrangement). Sensor 13 is arranged such that when the disk 15 is rotated, for example, in the direction indicated by two arrows in FIG. 5, it can measure lingual or labial strength in the vertical and lateral directions. Sensor 13 will measure strength in the downward direction when arranged in the 6:00 o'clock position. Sensor 13 will measure strength in the upward direction when arranged in the 12:00 o'clock position. The 3:00 o'clock position is for lateral rightward and the 9:00 o'clock position is for lateral leftward directions, respectively. The disk 15 may be pinned to disk 17 so that disk 15 can freely rotate. Because the disk can freely rotate, sensor 13 can be used to measure lingual or labial strength in any direction perpendicular to the direction of tongue extension. Retention for the aforementioned four measurement positions can be implemented (not shown) with a spring loaded locking pin and associated mating holes. The electrical connection for the sensors from the front disk 15 to the rear 17 may be made using slip rings (not shown). A person practiced in the art will be familiar with this connection technique. Disk 17 is mounted to the body of the handheld system 18, which can provide structural support and can be used for housing the electronics (alternatively, the handheld system can be cabled to a stationary unit with electronics). A grip 19 allows the Speech-Language Pathologist to hold the measurement system 18 and the chin support 16 provides a mechanism to hold the system steady and provide repeatable positioning for measurement sessions. The chin support can be implemented in a variety of ways to incorporate adjustments and may potentially be a disposable element in particular realizations of the invention.

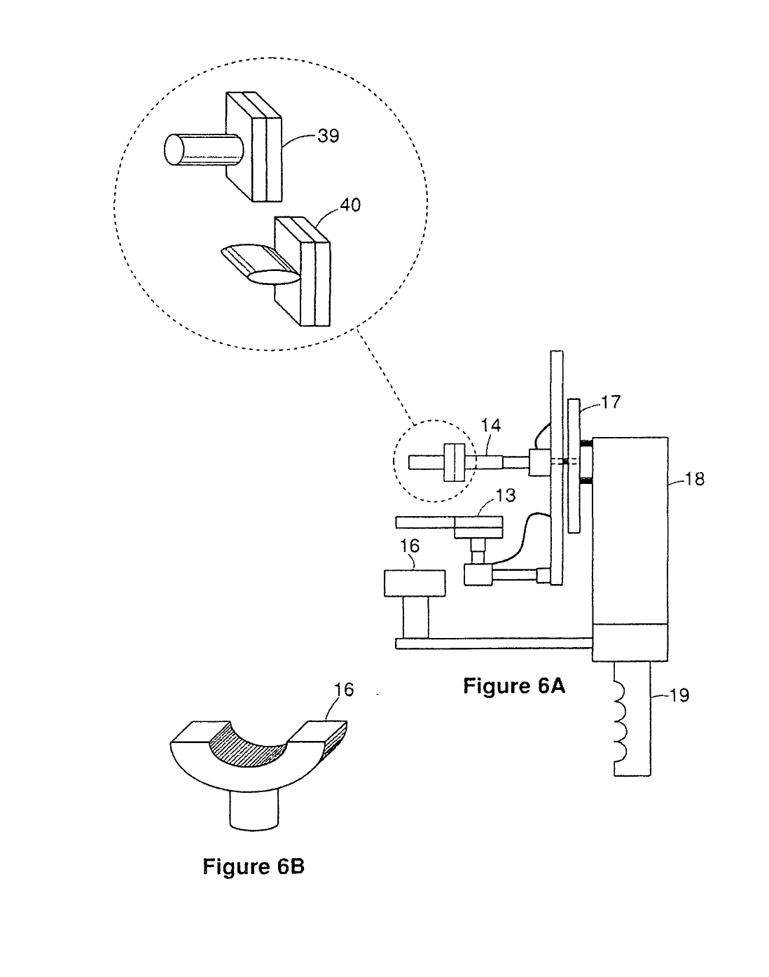

FIG. 6 shows the handheld in FIG. 5 with the labial seal disposables 39 and 40. The labial seal disposables have a shape that facilitates creating a labial seal. The shapes can be varied as a function of the preference of the therapist and the needs of the patient. Exemplary shapes are shown in FIG. 6.

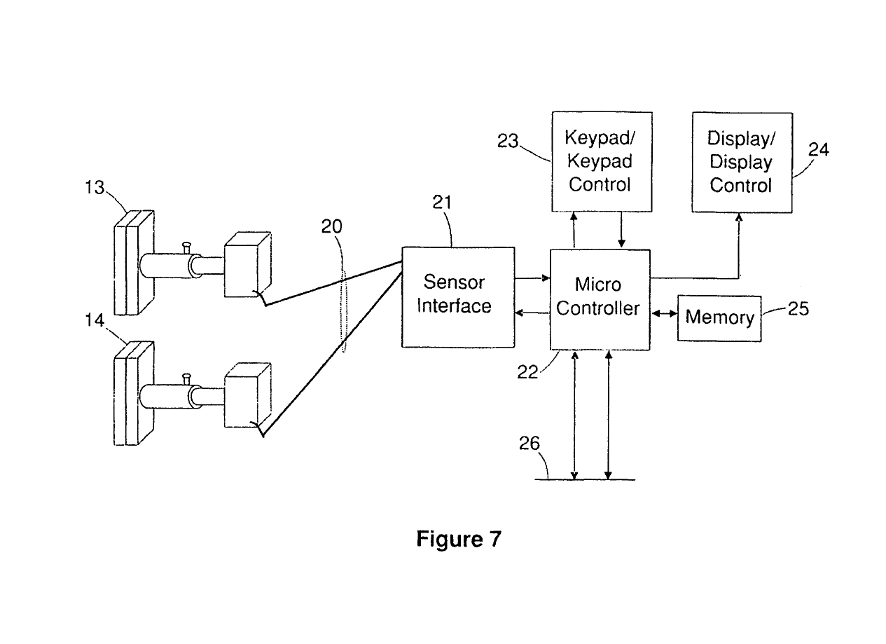

FIG. 7 shows the electronic subsystems associated with an embodiment of the invention. The sensors 13 and 14 are connected to the interface circuitry 21, through a cable harness 20. The sensor interface 21 is interfaced with a microcontroller 22. The microcontroller 22 is responsible for collecting the digitized force readings from the interface circuitry 21 and formatting that information for display on the display 24, or via the external interfaces 26. The microcontroller uses nonvolatile memory 25 to store persistent information such as sensor calibration information. A Keypad or keyboard 23 allows the operator to interact with the microcontroller, e.g., to input patient information and/or initiate test/therapy sessions. The external interfaces 26 can be implemented as a variety of standard interfaces (e.g., USB and RS-232) for the purpose of interfacing with a printer and generating printed reports of patient sessions, or for communication with a hospital database and keeping the test and patient information there.

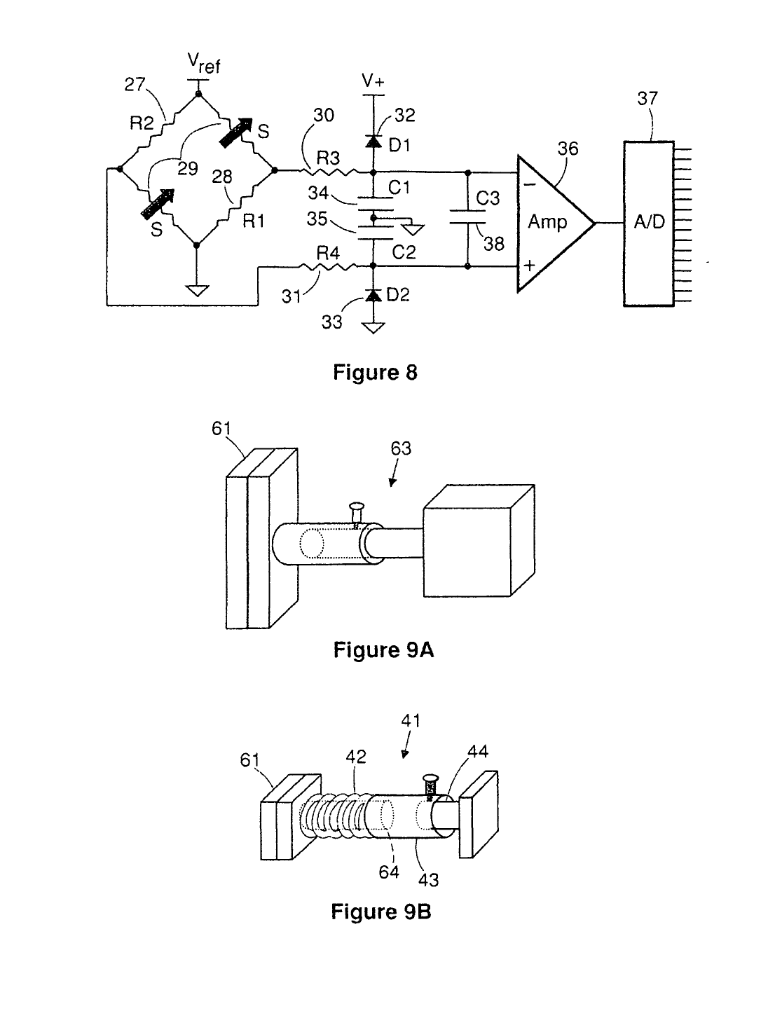

FIG. 8 shows the interface electronics that convert the signal from the sensor assembly 13 or 14 to a digital value which can be read by the microcontroller 22. In an embodiment of the invention, a force sensor 29 utilizes a pair of resistive arrays and resistors R2 27 and R1 28 to create differential bridge. Many sensors implement the full differential bridge as the sensor element. So the force sensor 29 is often included in sensor assembly 13 or 14. When a reference voltage is applied to the bridge, a differential voltage is created and presented to resistors R3 30 and R4 31. As force increases, the resistive elements 27 and 28 become more resistive and the differential voltage increases. Resistors R3 30 and R4 31 are used to provide current limiting and as a portion of a common mode lowpass filter. Each leg R3 30, C1 34 and R4 31, C2 35 (along with the thevanized impedance looking into the attached terminal of the bridge) dictate the time constant of the filter. Capacitor C3 38 along with the thevanized differential impedance creates a differential mode lowpass filter. The purpose of the filters is to limit the amount of noise picked up by the system that could cause extraneous readings. Diodes D1 32 and D2 33 provide input protection for amplifier 36 in the event that any high voltages are coupled into the sensor interface. Such a condition can occur due to an electric discharge from accumulated static buildup on a person or object when it comes in contact with the invention. Amplifier 36 is a differential amplifier that provides gain to the received differential voltage and converts it to a single ended voltage that can be converted to a digital reading by the analog/digital (A/D) converter 37.

The sensor interface does not require local adjustment to provide a zero level and gain adjustment. Rather, the microcontroller 22 can automatically establish a zero force point prior to a reading. As such the system is auto zeroing. The gain can be calibrated by applying a known force to the force sensor 29 and providing that reading to the microcontroller 22 through the keypad 23. The microcontroller can store the calibration in nonvolatile memory 25. In normal practice, the gain calibration should not vary significantly and should not require recalibration except on a prophylactic basis.

FIGS. 9A and 9B show embodiments of the present apparatus useful for rehabilitative therapy. A therapist may desire to use an isometric treatment with various extension or a resistive treatment with variable resistive forces as a mechanism to strengthen labial and lingual muscles. In FIG. 9A, an isometric assembly 63 similar to the sensor assembly (FIG. 3) without the force transducer 12 can provide an isometric treatment, in which the metal plate can create an isometric barrier. In FIG. 9B, a resistive assembly 41 can provide a variable resistance using a spring 42 in compression. The spring surrounds a guiding element 64, which may be a shaft as shown in the embodiment in FIG. 9B. The spring is connected to a tube 43 and the guiding element is captured in the tube from one end of the tube. The other end of the tube is connected to a shaft 44 via a pinning mechanism, so that the length of the shaft 44 can be adjusted.

Note that the sensor assembly in FIG. 3 can also be used to provide isometric treatment with or without the force transducer 12 present. If the isometric assembly is connected to a force transducer, electronic measurement of the force is also possible. The resistive assembly can be used in lieu of the sensor assembly or can be inserted into the sensor assembly, the rest of which has to be released from the force transducer, for a resistive session. In the latter case, the resistive assembly is connected to the force transducer.

It is possible to measure progress in the resistive therapy by noting and recording the linear compression of the spring indicating the degree of force. A set of linear markings can be used for this purpose. In general, the spring will provide a linear force as a function of compression as defined by Hooke's Law (F=−kx) where F is the force, x is linear distance and k is the spring constant. As such, a variety of devices with different spring constants can be used to achieve the desired level of resistive force. If the resistive assembly is connected to a force transducer, electronic measurement of the force is also possible. The disposable 61 used in isometric assembly 63 or resistive assembly 41 can either be disposable 6, disposable 6 with extension portion 6A, or disposable 39 or 40, depending on the kind of exercise desired. Similarly, assembly 63 or 41 can take the place of either assembly 13 or 14 in the handheld system shown in FIG. 5 or 6, depending on the kind of exercise desired.

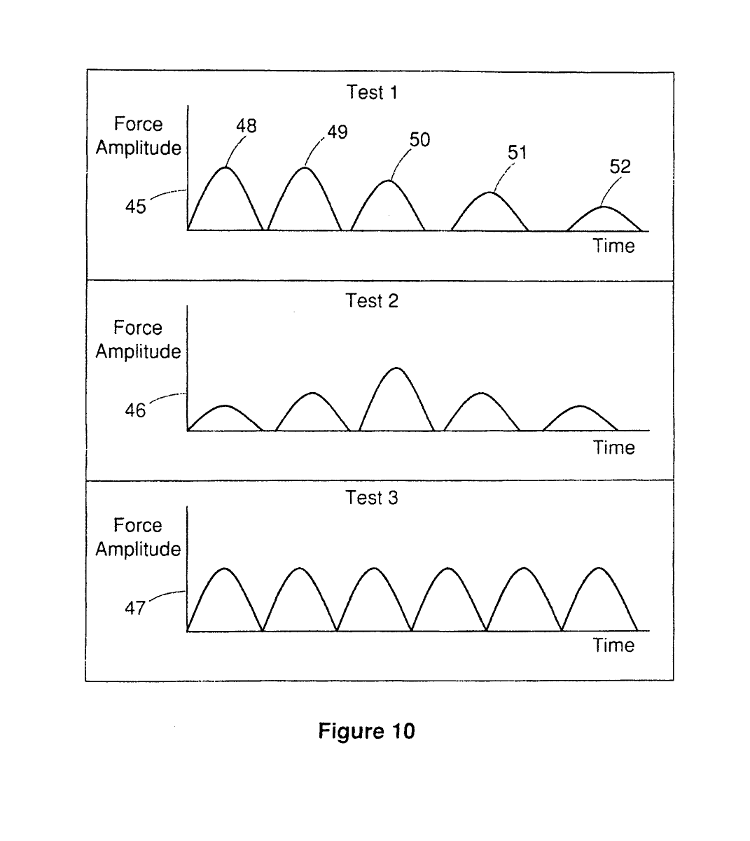

FIG. 10 shows graphically the results of 3 separate prototypical tests. The amplitude of the force is represented by the vertical (Y) axis and the elapsed time is represented by the horizontal (X) axis. The time scale pertains to a sequence of measurements associated with a test or a period of time during a therapy session. The curves associated with FIG. 10 are drawn in a relatively smooth and symmetric fashion; however, actual sessions can be more irregular. Graph 45 portrays a test or a therapy session with 5 tests or therapy repetitions. There are 5 curves (48, 49, 50, 51 and 52) in the test/therapy sequence. The curves are representative of the digital data captured by the invention during a test/therapy session. The X axis represents time associated with the samples and the Y axis is the measured amplitude of the force. The overall session and each of the curves provide information that is clinically significant.

In graph 45, there are 5 relative maximum amplitudes of the curves and 5 overall repetitions. In addition, the magnitude of the relative maximums decreases after curve 49. In this case, the session is showing that the patient was able to perform 5 repetitions, that the maximum amplitude of force is represented by curves 48 and 49, and that the patient began to fatigue after 49. So, the number of repetitions and the sequence of relative maximums of the amplitude of force provide a view of the most interesting contemporary clinical information. However, there is additional information that can be used and can provide clinical significance; some of them are the relative area under each curve, the shape of each curve, the number of repetitions in a period of time and the time during which a force greater than a certain amplitude is maintained by the patient. While this is not an exhaustive list, it does provide some indication of the type of information that the invention can provide and the type of data available to the clinician. Graph 46 shows a session with 5 repetitions and a pattern that would indicate that the patient was increasing the rigor of the session and subsequently fatiguing. Graph 47 shows 6 curves with relatively uniform maximum values indicating that the patient can reach a maximum of a certain amplitude of force and that there is no fatigue due to the exercise over the 6 repetitions.

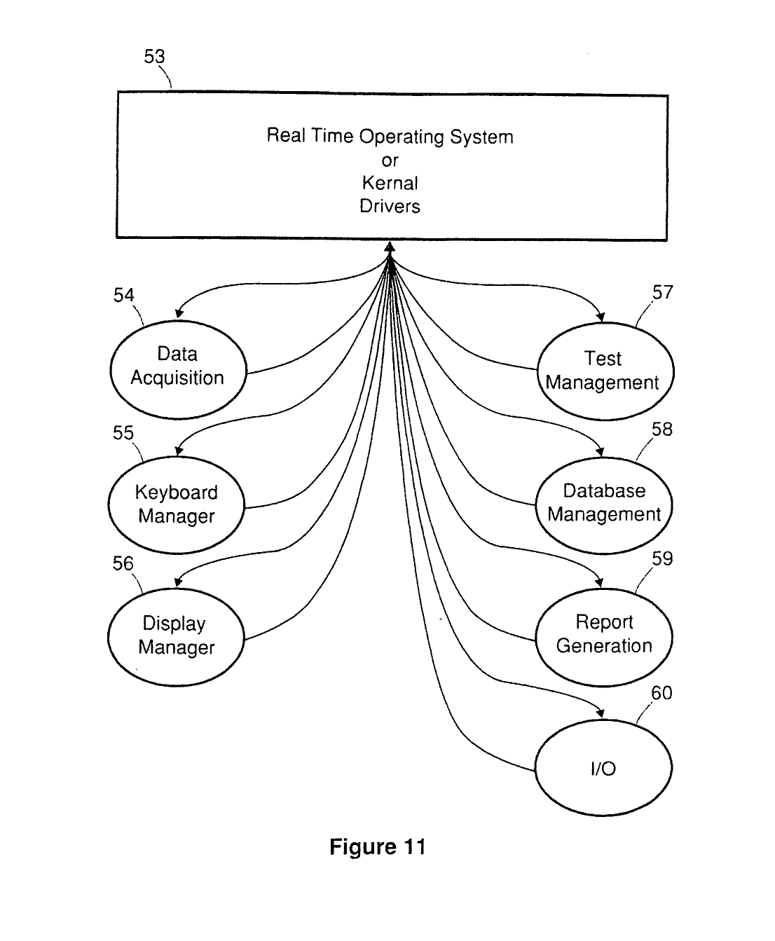

FIG. 11 shows a software architecture for an embodiment of the invention. A real time operating system (RTOS) or kernel 53 provides the basic software operational infrastructure. There are a number of available RTOS products available including Linux and VX-Works. In other embodiments, a simple kernel may be used as an alternative. The RTOS contains the drivers that support the hardware and constituent software modules. A data acquisition module 54 can handle the collection of data during a test or a therapy session. This module can acquire the data points from the hardware and provide digital processing. The digital processing may include digital filtration and algorithms to derive clinically significant information (such as relative maximums of the amplitude of force, repetitions, area under the curves). The keyboard manager 55 handles the management of local input from the keyboard. It can handle the input and interpretation of information from the keyboard. The keyboard can be used to input patient information, test information, and initiate test/therapy sequences. The keyboard manager can work in conjunction with modules such as the test management module 57 and the display manager 56.

The test management module 57 handles the sequencing and overall control of test/therapy sequences. It helps assure that patient data is entered and it will control the overall execution of tests and therapy sessions. The display manager 56 manages the display and the formatting of data to the display. It can operate under the control of the test management module 57. The database manager 58 handles the database within the instrument. The database can contain patient information, test information, test results, raw data, time of day, information about the therapy/therapist. The report generation module 59 is used to provide reports from test/therapy sessions in formats determined by the therapist or hospital. The reports can be printed out via an attached printer or downloaded for central storage or printing. The input/output (I/O) module 60 handles I/O to the invention. It can manage downloads of the database or reports. In addition, it can provide access to a central system to provide information for billing or uploads of information to be included in the reports.

While this invention has been particularly shown and described with references to example embodiments thereof, it will be understood by those skilled in the art that various changes in form and details may be made therein without departing from the scope of the invention encompassed by the appended claims.

1. Apparatus for measuring the strength or motility of a patient's tongue or lips, comprising:
a sensor assembly comprising:
a force-receiving element;
a force transducer providing a sensor signal; and
a shaft mechanically coupling a force applied to the force-receiving element by a patient's tongue or lips to the force transducer, wherein the length of the shaft is adjustable, thereby rendering the distance between the force-receiving element and the force transducer adjustable.

2. The apparatus of claim 1 wherein the force-receiving element is a plate.

3. (canceled)

4. The apparatus of claim 1 wherein the sensor assembly further comprises: a disposable element.

5. The apparatus of claim 4 wherein the disposable element is integral to the force-receiving element.

6. The apparatus of claim 4 wherein the disposable element is attachable to the force-receiving element.

7. The apparatus of claim 4 wherein the disposable element comprises:
an extension portion that can be inserted into the patient's mouth or adjacent to the patient's lips.

8. The apparatus of claim 4 wherein the disposable element comprises:
an extension portion having a shape that facilitates creation of a labial seal.

9. The apparatus of claim 1 further comprising:
electronics processing the sensor signal to produce an output signal.

10. The apparatus of claim 9 wherein the output signal comprises:
a profile of the amplitude of the force exerted by the patient's tongue or lips over time.

11. The apparatus of claim 1 wherein the sensor assembly is mounted to a disk so as to allow measurement in the direction of tongue extension.

12. The apparatus of claim 1 wherein the sensor assembly is mounted to a disk so as to allow measurement in any direction perpendicular to the direction of tongue extension.

13. The apparatus of claim 12 wherein the sensor assembly is mounted to a disk so as to allow measurement in lateral directions.

14. The apparatus of claim 12 wherein the sensor assembly is mounted to a disk so as to allow measurement in vertical directions.

15. The apparatus of claim 12 further comprising:
a second sensor assembly mounted to the disk so as to allow measurement in the direction of tongue extension.

16. Apparatus for providing therapy to increase the strength and motility of a patient's tongue or lips, comprising:
an assembly comprising:
a force-receiving element;
a support structure; and
a shaft connecting the force-receiving element to the support structure, wherein the length of the shaft is adjustable, thereby rendering the distance between the force-receiving element and the support structure adjustable.

17. The apparatus of claim 16 wherein the force-receiving element is a plate.

18. (canceled)

19. The apparatus of claim 16 wherein the assembly further comprises:
a disposable element.

20. The apparatus of claim 19 wherein the disposable element is integral to the force-receiving element.

21. The apparatus of claim 19 wherein the disposable element is attachable to the force-receiving element.

22. The apparatus of claim 19 wherein the disposable element comprises:
an extension portion that can be inserted into the patient's mouth or adjacent to the patient's lips.

23. The apparatus of claim 19 wherein the disposable element comprises:
an extension portion having a shape that facilitates creation of a labial seal.

24. The apparatus of claim 16 wherein the assembly further comprises:
a spring surrounding a guiding element attachable to the force-receiving element and captured in a tube, wherein the tube is connected to the shaft.

25. The apparatus of claim 24 wherein the assembly further comprises:
a set of linear markings measuring progress by noting the linear compression of the spring.

26. The apparatus of claim 16 wherein the assembly is mounted to a disk so as to provide an isometric barrier or a resistive force in the direction of tongue extension.

27. The apparatus of claim 16 wherein the assembly is mounted to a disk so as to provide an isometric barrier or a resistive force in any direction perpendicular to the direction of tongue extension.

28. The apparatus of claim 27 wherein the assembly is mounted to a disk so as to provide an isometric barrier or a resistive force in lateral directions.

29. The apparatus of claim 27 wherein the assembly is mounted to a disk so as to provide an isometric barrier or a resistive force in vertical directions.

30. The apparatus of claim 27 further comprising:
a second assembly mounted to the disk so as to generate an isometric barrier or a resistive force in the direction of tongue extension.

31. The apparatus of claim 16 wherein the assembly further comprises:
a force transducer providing a sensor signal.

32. The apparatus of claim 31 further comprising:
electronics processing the sensor signal to produce an output signal.

33. The apparatus of claim 32 wherein the output signal comprises:
a profile of the amplitude of the force exerted by the patient's tongue or lips over time.

34. A method of measuring the strength or motility of a patient's tongue or lips comprising:
contracting muscles of the tongue or the lips to exert a force on a force-receiving element disposed in front of the patient;
sensing the force exerted on the force-receiving element by the tongue or the lips to provide an electrical signal representative of the force;
deriving from the electrical signal a profile thereof over the duration of a test run, wherein the profile of the electrical signal is characteristic of the force exerted by the tongue or the lips over the duration of the test run; and
displaying and/or recording the profile of force.

35. The method of claim 34 wherein contracting the muscles of the tongue or the lips to exert force on the force-receiving element comprises:
pressing against the force-receiving element with the patient's tongue or lips.

36. The method of claim 35 wherein pressing against the force-receiving element with the patient's tongue or lips comprises:
extending the patient's tongue or lips forward.

37. The method of claim 35 wherein pressing against the force-receiving element with the patient's tongue or lips comprises:
moving the patient's tongue or lips in lateral directions.

38. The method of claim 35 wherein pressing against the metal force-receiving element with the patient's tongue or lips comprises:
moving the patient's tongue or lips in vertical directions.

39. The method of claim 34 wherein contracting the muscles of the tongue or the lips to exert force on the force-receiving element comprises the step of creating a labial seal.

40. A method of providing therapy to increase the strength and motility of a patient's tongue or lips comprising:
contracting muscles of the tongue or the lips to exert a force on a force-receiving element disposed in front of the patient and connected to a support structure by a shaft.

41. The method of claim 40 wherein the force-receiving element provides a resistive force and contracting the muscles results in displacement of the force-receiving element.

42. The method of claim 41 comprising:
noting the displacement of the force-receiving element.

43. The method of claim 40 wherein contracting the muscles of the tongue or the lips to exert force on the force-receiving element comprises:
pressing against the force-receiving element with the patient's tongue or lips.

44. The method of claim 43 wherein pressing against the force-receiving element with the patient's tongue or lips comprises:
extending the patient's tongue or lips forward.

45. The method of claim 43 wherein pressing against the force-receiving element with the patient's tongue or lips comprises:
moving the patient's tongue or lips in lateral directions.

46. The method of claim 43 wherein pressing against the force-receiving element with the patient's tongue or lips comprises:
moving the patient's tongue or lips in vertical directions.

47. The method of claim 40 wherein contracting the muscles of the tongue or the lips to exert force on the force-receiving element comprises:
creating a labial seal.

48. The method of 40 further comprising:
sensing the force exerted on the force-receiving element using a force transducer to provide an electrical signal representative of the force;
deriving from the electrical signal a profile thereof over the duration of a therapy session, wherein the profile of the electrical signal is characteristic of the force exerted by the tongue or the lips over the duration of the therapy session; and displaying and/or recording the profile of force.
